(19) United States
(12) Patent Application Publication
(10) Pub. No.: US 2021/0144944 A1
(43) Pub. Date: May 20, 2021

(54) SYSTEMS AND METHODS FOR THERMAL MANAGEMENT OF OUTDOOR ALGAE CULTIVATION

(71) Applicant: ExxonMobil Research and Engineering Company, Annandale, NJ (US)

(72) Inventors: Everett J. O'Neal, Asbury, NJ (US); Sarah E. Feicht, Raritan, NJ (US); Patrick L. Hanks, Bridgewater, NJ (US)

(21) Appl. No.: 17/082,082

(22) Filed: Oct. 28, 2020

Related U.S. Application Data
(60) Provisional application No. 62/935,689, filed on Nov. 15, 2019.

(51) Int. Cl. A01G 33/00; C12M 1/00

(52) U.S. Cl. CPC A01G 33/00; C12M 29/00; C12M 21/02

(57) ABSTRACT

Systems and methods for algae cultivation and, more particularly, to systems and methods for controlling algae cultivation water solution temperatures to optimize algae facility productivity. Effluent water from harvested algae water solutions is stored based on temperature conditions within storage reservoirs and recycled back into a cultivating algae solution to control the temperature thereof and enhance the growth and robustness of resultant algal biomass.

### CROSS-REFERENCE TO RELATED APPLICATIONS

This application claims the benefit of priority from U.S. Provisional Application No. 62/935,689 filed Nov. 15, 2019, the contents of which are herein incorporated by reference in their entirety.

### FIELD OF THE INVENTION

The present disclosure relates to algae cultivation and, more particularly, to systems and methods for controlling algae cultivation water solution temperature to optimize algae facility productivity.

### BACKGROUND OF THE INVENTION

Concerns about climate change, carbon dioxide (CO2) emissions, and depleting mineral oil and gas resources have led to widespread interest in the production of biofuels from algae, including microalgae. As compared to other plant-based feedstocks, algae have higher CO2 fixation efficiencies and growth rates, and growing algae can efficiently utilize wastewater, biomass residue, and industrial gases as nutrient sources.

Algae are photoautotrophic organisms that can survive, grow, and reproduce with energy derived entirely from the sun through the process of photosynthesis. Photosynthesis is essentially a carbon recycling process through which inorganic CO2 combines with solar energy, other nutrients, and cellular biochemical processes to output gaseous oxygen and to synthesize carbohydrates and other compounds critical to the life of the algae.

To produce algal biomass, algae cells are generally grown in a water solution comprising water and nutrients. The algae may be cultivated in indoor or outdoor environments, and in closed or open cultivation systems. Closed cultivation systems include photobioreactors, which utilize natural or artificial light to grow algae in an environment that is generally isolated from the external atmosphere. Such photobioreactors may be in a variety of shaped configurations, but are typically tubular or flat paneled. Open cultivation systems include natural and artificial ponds that utilize sunlight to facilitate photosynthesis. Artificial ponds are often shaped in circular or raceway (oval) configurations.

Various processing methods exist for harvesting cultivated algal biomass to extract lipids therefrom for the production of fuel and other oil-based products. Moreover, harvesting cultivated algal biomass can be used to produce non-fuel or non-oil-based products, including nutraceuticals, pharmaceuticals, cosmetics, chemicals (e.g., paints, dyes, and colorants), fertilizer and animal feed, and the like. However, algae growth and robustness depends on, among other things, the stability of the temperature of the water solution during cultivation. Vast fluctuations in water solution temperatures may hinder and/or be lethal to algae growth and, therefore, delay or otherwise impede algal biomass product production.

Because algal biomass produces valuable commodities, including sustainable biofuels and non-oil based products, control of water solution temperature fluctuations that may affect the quality and/or quantity of the biomass and downstream resultant products is desirable.

### SUMMARY OF THE INVENTION

The present disclosure relates to algae cultivation and, more particularly, to systems and methods for controlling algae cultivation water solution temperature to optimize algae facility productivity.

In some aspects, a system is disclosed and includes a cultivation vessel containing an algae water solution for cultivation thereof. A first reservoir is in fluid connection with the cultivation vessel to receive and store a first effluent water derived from the algae water solution, and optionally a second reservoir is in fluid communication with the cultivation vessel to receive and store a second effluent water derived from the algae water solution. A first effluent water supply line fluidly couples the first reservoir to the cultivation vessel to deliver the stored first effluent water to the cultivation vessel, and optionally a second effluent water supply line fluidly couples the second reservoir to the cultivation vessel to deliver the stored second effluent water to the cultivation vessel. The first and second effluent waters may each be, independently, a daytime effluent water or a nighttime effluent water.

In some aspects, a method is disclosed and includes the steps of cultivating an algae water solution within a cultivation vessel, extracting a first effluent water from the cultivation vessel, diverting the first effluent water to a first reservoir, and optionally extracting a second effluent water from the cultivation vessel, and diverting the second effluent water to a second reservoir A temperature of the algae water solution is controlled by delivering the first effluent water to the cultivation vessel and optionally delivering the second effluent water to the cultivation vessel.

### BRIEF DESCRIPTION OF THE DRAWINGS

The following figures are included to illustrate certain aspects of the present disclosure, and should not be viewed as exclusive examples. The subject matter disclosed is capable of considerable modifications, alterations, combinations, and equivalents in form and function, without departing from the scope of this disclosure.

FIG. 1 is a plot showing an example temperature trend of an actual algae water solution as compared to actual ambient temperature during algal biomass cultivation.

FIG. 2 is a schematic diagram of an example temperature control system for cultivating algal biomass, according to one or more aspects of the present disclosure.

### DETAILED DESCRIPTION OF THE INVENTION

The present disclosure relates to algae cultivation and, more particularly, to systems and methods for controlling algae cultivation water solution temperature to optimize algae facility productivity.

Biofuel production from cultivated algae solutions offers sustainable energy solutions to reduce reliance on fossil fuels and reduce greenhouse gas emissions. Other non-oil-based products can additionally be derived from algal biomass, including nutraceuticals, pharmaceuticals, cosmetics, chemicals (e.g., paints, dyes, and colorants), fertilizer and animal feed, and the like. To accomplish substantial economic, environmental, and societal impact, cultivated algae must be of sufficient quality and in sufficient quantity for harvesting and processing. Moreover, production of healthy algal biomass at reduced energy and operational expenditures allows algae-derived fuels and other non-oil-based products to become more cost-effective for production by algae facilities and, thus, more widely available to the public.

Algae cells are typically cultivated in algae solutions that may be upwards of hundreds to thousands of liters or more in volume, depending on the type and configuration of the particular cultivation system. Algae cells are generally cultivated for a predetermined period of time and allowed to reach a particular desired concentration in the algae solution (e.g., about 500 to 1000 parts per million) before the algal biomass is harvested. Depending on the type and configuration of the particular cultivation system, the predetermined cultivation time may be about one day to about one or more weeks. Accordingly, the amount of algal biomass for harvesting may be relatively dilute compared to the total volume of the algae solution. Because algae growth and robustness depends critically on the temperature of the algae solution during cultivation, certain cultivation temperatures may be too high or too low, thereby hindering or being lethal to the production of quality algal biomass. Moreover, the majority of algae cultivation systems are located in outdoor algae facilities, whereby daytime and nighttime conditions, as well as other weather phenomena (e.g., wind conditions) exacerbate algae solution temperature fluctuations. Indeed, algae facilities are typically located in non-agricultural lands that are exposed to high solar intensity (having little to no foliage ground coverage or over coverage), such as arid or desert lands, with relatively high maximum daytime temperatures (e.g., about 35° C. to 40° C., or higher) and relatively low minimum nighttime temperatures (e.g., about 0° C. to 15° C., or lower).

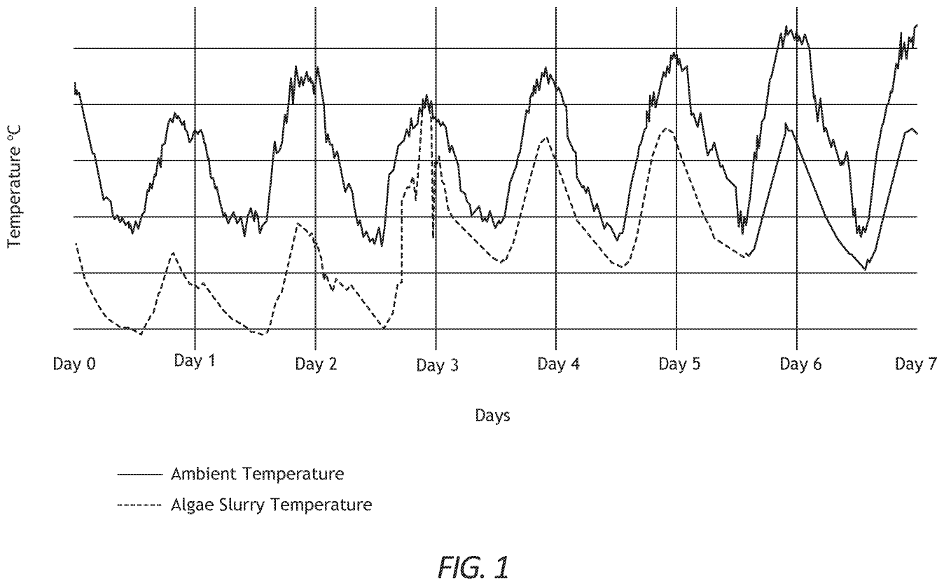

For illustration, FIG. 1 shows a plot trend of the measured temperature of an actual algae water solution compared to measured ambient temperature during algal biomass cultivation. As shown, as ambient temperature rises and falls, so does the temperature of the algae solution. These thermal fluctuations of the algae solution can be detrimental to the cultivating algae cells therein, and further limit the particular strain(s) of algae cells that may be utilized and the location(s) of particular algae facilities. Indeed, temperatures that are greater than about 30° C. can be lethal to certain strains of algae cells. Temperatures greater than about 35° C. or 40° C. and less than about 10° C. can be lethal to many algae cells currently in use for producing algal biomass. As shown in FIG. 1, for the particular algae solution observed, the solution temperature peaks over 30° C. on multiple days (i.e., Days 3-7). Temperatures that approach equal to or less than about 10° C. or equal to or greater than about 40° C. may negatively affect the growth of the algae cells, reducing the productivity of the algae facility.

Open cultivation systems may experience some cooling due to evaporation, but depending on the location of the outdoor cultivation system, evaporation may not be ample to maintain desired algae cultivation temperature ranges. However, whether using open or closed cultivation systems, traditional cultivation methods often control temperature fluctuations by utilizing power-driven processes (e.g., natural gas power plants) to heat and/or cool an algae water solution, which are particularly expensive in terms of cost, energy consumption, and facility footprint requirements, especially given the large volumes of algae solution that must be temperature controlled for large-scale algal biomass production.

The present disclosure provides systems and methods that harness, store, and utilize solar thermal energy for solar powered control of algae water solution temperatures during algae cultivation. More particularly, a pump-driven (e.g., solar or electric hydraulic) process is described in which at least two thermal reservoirs (referred to herein simply as “reservoir(s)”) are used to store excess solar thermal energy in the form of effluent water of different temperatures (due to solar exposure) derived from a harvested algae water solution, wherein the stored effluent water can be recycled within a cultivation system throughout a cultivation period. That is, at least two separate reservoirs are used to store hot, daytime effluent water during high-temperature daytime hours and cold, nighttime effluent water during low-temperature nighttime hours. When the daytime (hot) and nighttime (cold) effluent water is not being stored within the reservoirs, for example, it may be recycled back into a cultivating algae solution to control temperature. Accordingly, the present disclosure allows the temperature of an algae solution to be at least partially controlled and maintained within a desired range while reducing associated energy consumption and other costs, promoting thermal efficiency, and thereby optimizing the productivity of an algae production facility. Moreover, while the present disclosure is directed primarily to storing and utilizing solar thermal energy, the systems and methods described herein may additionally be run on electricity derived through solar energy.

The systems and methods described herein advantageously harness the natural, ambient temperature fluctuations of existing algae facilities and can be integrated (i.e., in fluid communication with one or more components) therewith without substantial equipment or structural changes or additions to such facilities. Indeed, algae facilities are equipped with multiple pipes and pumps of various sizes and capacities, typically large pipes and pumps capable of facilitating flow of flowable materials (e.g., algae solutions, water, and the like) therethrough. A significant fraction of solar energy used to run the algae facility may be stored in one or more of the reservoirs described herein. Such reservoirs may be located, for example, at an elevated or highest elevated location in the algae facility (e.g., at the top of the facility, such as on a top of a hill or adjacent structure). For example, a hypothetical algae facility designed to produce 10 thousands of barrels per day of algae-derived biofuels and located near a shoreline may be approximately 40 square miles in size and have a highest (peak) elevation of about 100 to about 200 feet (about 30.5 meters to about 61 meters) above sea level. One or more of the reservoirs described herein may be preferably positioned at such elevation, or other elevated locations relative to a cultivation vessel(s), in order to utilize gravity to feed the cultivation vessel(s) with daytime or nighttime effluent water, thereby further facilitating energy and other cost savings associated with the systems and methods described herein. Although one or more reservoirs used in the systems and methods described herein may be positioned at an elevated location within an algae facility in some instances, the reservoirs described herein may alternatively be positioned at any other location within an algae facility to aid in controlling algae solution temperatures, without departing from the scope of the present disclosure.

The systems and methods described herein are particularly applicable to algae solutions that have short cultivation residence times (i.e., the time in which the algae solution is within a cultivation vessel is relatively short). For example, optimal cultivation times may be less than or equal to about 2 days, including about 1.5 days, about 1 day, and the like. Indeed, the present disclosure can result in reduced cultivation times for algae to reach desired concentrations by controlling temperature fluctuations. In view of the present disclosure, calculations demonstrate that reservoirs storing daytime and nighttime effluent water with 25%, for example, of a hold-up volume that would otherwise make up a complete algae solution, can mitigate thermal fluctuations of said algae solution by at least about 5° C. in either direction of preference to optimize algae facility production. For example, the temperature direction of preference may change season-to-season. Accordingly, short cultivation residence times are particularly applicable to the present disclosure in order to realize substantial benefit from tuning cultivating algae solution temperatures within desired ranges using stored daytime and nighttime effluent water. Over longer cultivation residence times (e.g., greater number of 24-hour periods), the thermal fluctuations within the cultivation vessel(s) begin to once again mimic ambient temperature, rather than benefit from the temperature control by input of recycled effluent water. As such, algae facilities employing the systems and methods of the present disclosure may be preferably operated with a focus on short cultivation residence times (and, thus, small algae concentration changes), which may benefit from continuous or semi-continuous cultivation systems, for example.

As used herein, the term “algae solution” or “algae water solution,” and grammatical variants thereof, refers to a flowable liquid comprising at least water, algae cells, and algae nutrient media (e.g., phosphorous, nitrogen, and optionally additional elemental nutrients). The algae slurries described herein may have an algae concentration of less than about 0.2%, and typically have a viscosity similar to water.

Algal sources for preparing the algae solution include, but are not limited to, unicellular and multicellular algae. Examples of such algae can include, but are not limited to, a rhodophyte, chlorophyte, heterokontophyte, tribophyte, glaucophyte, chlorarachniophyte, euglenoid, haptophyte, cryptomonad, dinoflagellum, phytoplankton, and the like, and combinations thereof. In some examples, algae can be of the classes Chlorophyceae and/or Haptophyta. Specific species can include, but are not limited to, Neochloris oleoabundans, Scenedesmus dimorphus, Euglena gracilis, Phaeodactylum tricornutum, Pleurochrysis carterae, Prymnesium parvum, Tetraselmis chui, and Chlamydomonas reinhardtii. Additional or alternate algal sources can include one or more microalgae of the Achnanthes, Amphiprora, Amphora, Ankistrodesmus, Asteromonas, Boekelovia, Borodinella, Botryococcus, Bracteococcus, Chaetoceros, Carteria, Chlamydomonas, Chlorococcum, Chlorogonium, Chlorella, Chroomonas, Chrysosphaera, Cricosphaera, Crypthecodinium, Cryptomonas, Cyclotella, Dunaliella, Ellipsoidon, Emiliania, Eremosphaera, Ernodesmius, Euglena, Franceia, Fragilaria, Gloeothamnion, Haematococcus, Halocafeteria, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Phaeodactylum, Phagus, Pichochlorum, Pseudoneochloris, Pseudostaurastrum, Platymonas, Pleurochrysis, Pleurococcus, Prototheca, Pseudochlorella, Pyramimonas, Pyrobotrys, Scenedesmus, Schizochlamydella, Skeletonema, Spyrogyra, Stichococcus, Tetrachlorella, Tetraselmis, Thalassiosira, Tribonema, Vaucheria, Viridiella, and Volvox species, and/or one or more cyanobacteria of the Agmenellum, Anabaena, Anabaenopsis, Anacystis, Aphanizomenon, Arthrospira, Asterocapsa, Borzia, Calothrix, Chamaesiphon, Chlorogloeopsis, Chroococcidiopsis, Chroococcus, Crinalium, Cyanobacterium, Cyanobium, Cyanocystis, Cyanospira, Cyanothece, Cylindrospermopsis, Cylindrospermum, Dactylococcopsis, Dermocarpella, Fischerella, Fremyella, Geitleria, Geitlerinema, Gloeobacter, Gloeocapsa, Gloeothece, Halospirulina, Iyengariella, Leptolyngbya, Limnothrix, Lyngbya, Microcoleus, Microcystis, Myxosarcina, Nodularia, Nostoc, Nostochopsis, Oscillatoria, Phormidium, Planktothrix, Pleurocapsa, Prochlorococcus, Prochloron, Prochlorothrix, Pseudanabaena, Rivularia, Schizothrix, Scytonema, Spirulina, Stanieria, Starria, Stigonema, Symploca, Synechococcus, Synechocystis, Tolypothrix, Trichodesmium, Tychonema, and Xenococcus species. Any combination of the aforementioned algae sources may additionally be used to prepare an algae solution.

The water for use in preparing the algae solution may be from any water source including, but not limited to, fresh water, brackish water, seawater, wastewater (treated or untreated), synthetic seawater (e.g., water with added salts), and any combination thereof.

The algae nutrient media for use in forming an algae solution may comprise at least nitrogen (e.g., in the form of ammonium nitrate or ammonium urea) and phosphorous. Other elemental micronutrients may also be included, such as potassium, iron, manganese, copper, zinc, molybdenum, vanadium, boron, chloride, cobalt, silicon, and the like, and any combination thereof.

As used herein, the term “cultivation vessel,” “vessel,” and grammatical variants thereof, refers to any of an open or closed algae cultivation system used for the growth of algal biomass, including bioreactors, photobioreactors, natural ponds, artificial ponds (e.g., raceway ponds), and the like.

As used herein, the term “daytime,” and grammatical variants thereof, refers to any time within a 24-hour period between sunrise and sunset in the particular geographical location where the cultivation vessel is located, and reckoned from one midnight hour to the next. For example, the “daytime effluent water” described herein may be derived from an algae water solution at any time between sunrise and sunset, including the entirety of said duration or a subset thereof (e.g., only during one or more hours in which the daytime temperature is highest). Conversely, the term “nighttime,” and grammatical variants thereof, refers to any time within a 24-hour period between sunset and sunrise in the particular geographical location where the cultivation vessel is located, and reckoned from one midnight to the next. For example, the “nighttime effluent water” described herein may be derived from an algae water solution at any time between sunset and sunrise, including the entirety of said duration or a subset thereof (e.g., only during one or more hours in which the nighttime temperature is lowest). In some instances, daytime effluent water may be collected from a cultivation vessel(s) (derived from an algae solution) at temperatures of about 30° C. to about 40° C. during the daytime hours, and nighttime effluent water may be collected from a cultivation vessel(s) (derived from an algae solution) at temperatures of about 10° C. to about 20° C. during nighttime hours, although other temperatures outside of these ranges are also within the scope of the present disclosure.

Various aspects of the present disclosure are described with reference to addressing concerns associated with thermal fluctuations of algae solutions during cultivation within closed cultivation systems, such as photobioreactors. Typically, closed cultivation systems are not subject to evaporative cooling, as is the case for open cultivation systems (e.g., outdoor raceway ponds) and, thus, closed cultivation systems may comparatively subject an algae solution to greater thermal fluctuations. However, it is to be appreciated that any or all aspects described herein may be equally applicable to open cultivation systems, without departing from the scope of the present disclosure. Indeed, certain environments, such as the Gulf Coast of the United States, exhibit rather large ambient temperature fluctuations between daytime and nighttime compared to other environments, which can negatively influence algae solution temperature; thus, outdoor cultivation systems in such environments may beneficially utilize any or all aspects of the present disclosure.

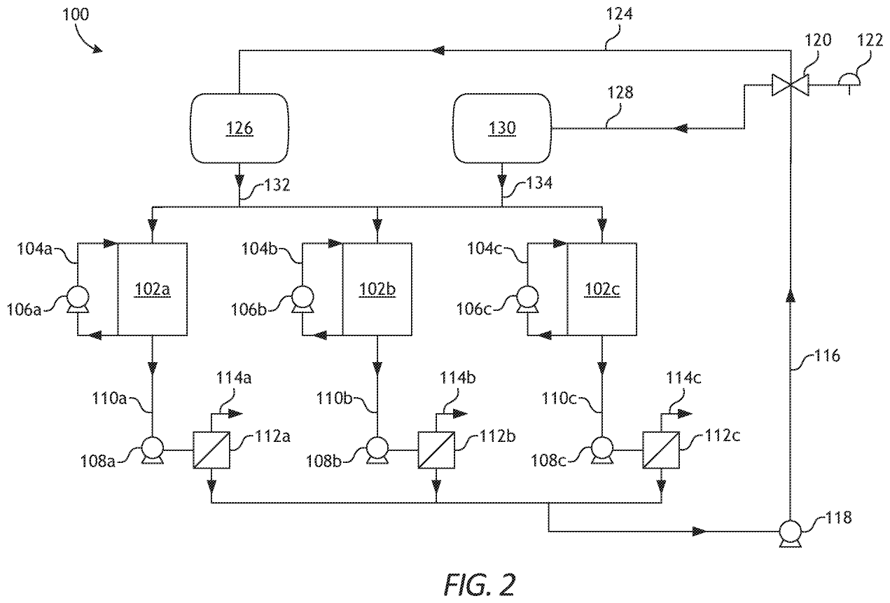

FIG. 2 is a schematic diagram of an example temperature control system 100 for cultivating algal biomass, according to one or more aspects of the present disclosure. The system 100 comprises closed algae cultivation vessels 102a, 102b, and 102c. The closed cultivation vessels 102a, b, c may be, for example, tubular photobioreactors, or any other types of closed cultivation vessels, and may be of the same or different types. It is to be appreciated that while three cultivation vessels 102a, b, c are shown in FIG. 2, one, two, or greater than three cultivation vessels may alternatively be used in accordance with the aspects of the present disclosure. Moreover, as provided above, the cultivation vessel(s) need not be closed cultivation vessel(s) and may be, instead, one or more open cultivation vessel(s) or a combination of closed and open cultivation vessels, without departing from the scope of the present disclosure.

Algae solution is contained within each of the cultivation vessels 102a, b, c, and cultivated by circulation of the algae solution therewithin. In some instances, the algae solution is circulated using one or more recirculation conduits (e.g., pipes, hoses, channels, troughs, and the like) and a pump with suitable valving, if necessary. For example, as shown in FIG. 2, recirculation conduits 104a, 104b, and 104c utilize pumps 106a, 106b, and 106c to recirculate the algae solution within cultivation vessels 102a, b, c, respectively. Alternatively or in addition to a recirculation conduit and pump, a gas (e.g., air, CO2, and the like) sparger or other injector (not shown) may be used to achieve lift and circulation of the algae cells based on gas separation. The recirculation conduits 104a, b, c , or other conduits, may additionally be used to seed new algae solutions into the cultivation vessels during or after final harvesting of prior cultivated algae solutions, in accordance with various aspects of the present disclosure.

With continued reference to FIG. 2, after the algae solution is cultivated for a predetermined period of time within cultivation vessels 102a, b, c, it is harvested using one or more harvesting systems. Harvesting systems may include one or more components configured to achieve separation of algal biomass from at least a portion of the remaining components of an algae solution (i.e., separate comparatively concentrated algal biomass from the remaining water and nutrients within an algae solution). Examples of such harvesting components may include, but are not limited to, a skimmer (e.g., weir skimmer), a membrane filter (or membrane module comprising one or more membranes), a centrifuge, and an air sparger, often coupled to pumps, one or more conduits (e.g., pipes, hoses, channels, troughs, and the like), and suitable valving. Any combination of the aforementioned harvesting components may be used alone or in combination, along with other known harvesting components, as appropriate. As shown in FIG. 2, pumps 108a, 108b, and 108c pump the algae solution through harvesting conduits 110a, 110b, and 110c from cultivation vessels 102a, b, c, respectively. Algal biomass is concentrated in harvesting systems 112a, 112b, and 112c, which may be any type of harvesting system, including those described above, and removed from the system 100 through biomass conduits 114a, 114b, and 114c for further processing (e.g., dewatering and extracting of lipids for biofuel production).

The remaining component of the algae solution after harvesting of the algal biomass in harvesting systems 112a, b, c is primarily effluent solution water (with any additional remaining algae nutrients not consumed by the algae cells), which is collectively referred to herein as “effluent water.” According to the present disclosure, this effluent water does not flow into the biomass conduits 114a, b, c and is not otherwise discarded. Rather, the effluent water from the harvesting systems 112a, b, c is flowed into effluent water conduit 116. Effluent water conduit 116 is represented as a single conduit in which effluent water from each of harvesting systems 112a, b, c is flowed; however, it is to be appreciated that greater than one, including a separate or multiple separate effluent water conduits for each harvesting system, may be employed, without departing from the scope of the present disclosure. Effluent conduit 116 is equipped with one or more pumps 118 (one shown) to facilitate flow of the effluent water from the harvesting systems 112a, b, c and through effluent conduit 116.

Advantageously, and as described herein, the aspects of the present disclosure encourage energy and cost savings by integrating hydroelectric storage of effluent water to assist in controlling the temperature of a cultivating algae solution. The effluent water conduit 116 collects effluent water at one or more times during daytime or nighttime hours, as defined herein above. Therefore, the temperature of the effluent water collected during daytime hours (the “daytime effluent water”) will generally have a relatively increased temperature compared to effluent water collected during nighttime hours (the “nighttime effluent water”). For example, in one or more aspects, daytime effluent water is collected during the hottest six hours of the daytime and nighttime effluent water is collected during the coldest six hours of the nighttime; the remaining 12 hours of the 24-hour period is used to input either the daytime effluent water or the nighttime effluent water (or both) into the cultivation vessels 102a, b, c to control cultivating algae solution temperatures therein within a desired range, as described below.

The effluent water, whether daytime effluent water or nighttime effluent water, flows through the effluent water conduit 116 to valve 120. The valve 120 may be configured to selectively divert the effluent water to two or more destinations based on the measured temperature of the effluent water (e.g., by use of a servo or other type of motor or other drive mechanism). More specifically, the valve 120 may be equipped or otherwise in communication with a temperature sensor 122, which measures the temperature of the effluent water in effluent water conduit 116, thereby being able to distinguish between daytime effluent water and nighttime effluent water. The particular temperature range(s) defining the daytime and nighttime effluent water may be dependent, among other factors, on the location of the algae facility (i.e., the ambient temperatures at said location), the season, other weather conditions, and the like. As such, the temperature sensor 122 may be programmable, and re-programmable, to define the particular temperature ranges of daytime and nighttime effluent water, depending on specific circumstances. If the temperature sensor 122 detects a temperature consistent with daytime effluent water, valve 120 may be operated to direct (divert) the daytime effluent water through daytime water conduit 124 and into a daytime reservoir 126. Alternatively, if the temperature sensor 122 detects a temperature consistent with nighttime effluent water, valve 120 may be operated to direct (divert) the nighttime effluent water through nighttime water conduit 128 and into a nighttime reservoir 130.

Valve 120 need not be equipped with temperature sensor 122, and may include a different type of sensor for distinguishing between daytime and nighttime effluent water or may not be equipped with a sensor at all. For example, valve 120 may be equipped with a light (sunlight) sensor to distinguish between daytime and nighttime effluent water, or may be equipped with a timer to switch the valve automatically between daytime hours and nighttime hours, and the like, and any combination thereof. Alternatively, valve 120 may be manually operable, and adjusted by operators at the algae facility to divert daytime and nighttime effluent water to the appropriate conduit and reservoir. Other configurations for controlling the diversion of daytime and nighttime effluent water to the appropriate conduit and reservoir are also contemplated within the scope of the present disclosure.

Daytime reservoir 126 and nighttime reservoir 130 may be at any location relative to one another and need not be in any particular order, need not be located on the same relative side of an algae facility, and the like. The particular location and configuration of the daytime reservoir 126 and nighttime reservoir 130 may depend on a number of factors, including the configuration of the algae facility, the available space within an algae facility, and the like, and combinations thereof. Moreover, the daytime reservoir 126 and nighttime reservoir 130 need not be of the same shape or size, provided that they each have sufficient capacity to store their respective effluent water volumes, and may be closed reservoirs (e.g., drums, barrels, and the like) or open reservoirs (e.g., ponds, pools, and the like). In some examples, the reservoirs 126, 130 may be cylindrical, polygonal, ovoid, triangular, irregular shaped, and the like, and have any suitable volume capacity given the particular algae facility. In some examples, the reservoirs have a volume in the range of about 1 million gallons to about 250 million gallons, encompassing any value and subset therebetween. There may be many such reservoirs at a site, with their total volume being in the range of about 10% to about 100% of the existing algae cultivation vessel (open or closed) volume. Further, as provided above, greater than two reservoirs may be used in accordance with the present disclosure, including greater than one daytime reservoir and greater than one nighttime reservoir, and the volume of each such reservoir need not be equal. For example, in various aspects, a greater gradation of diverted effluent water temperatures may be separated using multiple reservoirs (e.g., the daytime and nighttime effluent water is diverted to multiple reservoirs based on multiple temperature ranges within each of the daytime and nighttime hours). It is also contemplated that the daytime reservoir 126 and nighttime reservoir 130 could be the same vessel in some examples, and that this single vessel could be filled and then completely emptied of daytime effluent water, then filled and completely emptied of nighttime effluent water, with this process repeating at appropriate intervals.

In one or all aspects, daytime reservoir 126 and/or nighttime reservoir 130 may be insulated. Such insulation may ensure that the temperature of the daytime effluent water and nighttime effluent water within reservoirs 126, 130, respectively, are maintained or are only minimally altered during storage. Moreover, one or more temperature sensors may be included within reservoirs 126, 130 to assess the precise temperature of the stored effluent water prior to delivering the effluent water to one or more cultivation vessels, as described below, to better control the temperature of the algae solution therein. In one or any aspects, multiple temperature sensors may be used within the reservoirs 126, 130 to assess temperature at various depths within the effluent water column, for additional temperature control.

In example operation of the system 100, if the temperature of the cultivating algae solution in one or all of cultivation vessels 102a, b, c has dropped or is predicted or otherwise expected to drop below a predetermined low temperature (e.g., below 10° C.), stored daytime water effluent from daytime reservoir 126 may be flowed through daytime temperature control conduit 132 and delivered to one or all of cultivation vessels 102a, b, c. Alternatively, if the temperature of the cultivating algae solution in one or more of cultivation vessels 102a, b, c has increased or is predicted or otherwise expected to increase above a predetermined high temperature (e.g., above 30° C.), stored nighttime effluent water from nighttime reservoir 130 is flowed through nighttime temperature control conduit 134 and delivered to one or more of cultivation vessels 102a, b, c. In various aspects, the stored daytime and nighttime effluent water may be flowed through daytime temperature control conduit 132 and nighttime temperature control conduit 134 simultaneously to fill the reservoirs 126, 130 or at a time in which the reservoirs 126, 130 are not being filled (i.e., no influx of effluent water is being received by the reservoirs 126, 130). In some aspects, as shown, daytime and nighttime temperature control conduits 132, 134 may not be equipped with pumps, as they may instead utilize head pressure and gravity flow for delivery of the stored daytime and nighttime effluent water from reservoirs 126, 130. However, it is within the scope of the present disclosure to equip daytime and nighttime temperature control conduits 132, 134 with one or more pumps or other equipment to facilitate flow of the stored daytime and nighttime effluent water.

The particular configuration of the various components of system 100 in FIG. 2 is non-limiting and thus not restricted to the configuration shown in FIG. 2. Where not shown, each of the various conduits may be equipped with one or more valves or other flow control devices to block flow, allow flow, divert flow, or otherwise control flow of a flowable material within the conduits. Each of the described pumps, and any additional pumps used in connection with the systems and methods of the present disclosure, may be any type of pump capable of facilitating the flow of a flowable material through an associated conduit. Particular pumps for use in aspects of the present disclosure include any type of known hydraulic or positive displacement pump, and such pumps may be powered using electricity derived from solar energy.

Accordingly, the system 100 of FIG. 2 provides for methods of controlling the temperature of a contained and cultivating algae solution within one or more cultivation vessels. Based on the system described in FIG. 2, the methods include cultivating the algae solution, harvesting algal biomass therefrom and redirecting or flowing effluent water from the remaining algae solution components (e.g., mostly water) through a conduit and into either a daytime or nighttime reservoir, depending on the temperature of the effluent water (which correlates to whether the effluent water was collected during the daytime or the nighttime). The effluent water is stored in the appropriate reservoir and, depending on the temperature needs of the cultivating algae solution, either the daytime or nighttime effluent water is delivered through a conduit to the one or more cultivation vessels to control temperature of the cultivating water solution therein.

While the system 100 of FIG. 2 uses effluent water to both increase and decrease the temperature of the algae solutions within cultivation vessels 102a, b, c, a system could readily be designed that performed only one of these functions. For example, a system within the scope of this disclosure could divert and store only daytime effluent water for use to increase the temperature of the algae solutions within cultivation vessels 102a, b, c, during the night (e.g., or divert and store only nighttime effluent water for use during the day).

### EXAMPLES LISTING

The present disclosure provides, among others, the following examples, each of which may be considered as optionally including any alternate example.

Clause 1. A system comprising: a cultivation vessel containing an algae water solution for cultivation; a first reservoir in fluid communication with the cultivation vessel to receive and store a first effluent water derived from the algae water solution; optionally a second reservoir in fluid communication with the cultivation vessel to receive and store a second effluent water derived from the algae water solution; a first effluent water supply line fluidly coupling the first reservoir to the cultivation vessel to deliver the stored first effluent water to the cultivation vessel; and optionally a second effluent water supply line fluidly coupling the second reservoir to the cultivation vessel to deliver the stored second effluent water to the cultivation vessel.

The first effluent water may be a daytime or a nighttime effluent water, and the second effluent water may also be a daytime or nighttime effluent water.

Clause 2. The system of Clause 1, wherein the cultivation vessel is a closed or open cultivation vessel.

Clause 3. The system of Clause 1 or 2, wherein the cultivation vessel is a photobioreactor.

Clause 4. The system of any of the preceding Clauses, wherein the first effluent water supply line delivers the stored first effluent water from the first reservoir to the cultivation vessel to affect a temperature of the algae water solution.

Clause 5. The system of any of the preceding Clauses, wherein the first or second effluent water supply line delivers the stored first or second effluent water from the first or second reservoir to the cultivation vessel to increase a temperature of the algae water solution above about 10° C.

Clause 6. The system of any of the preceding Clauses, wherein the second effluent water supply line delivers the stored second effluent water from the second reservoir to the cultivation vessel to affect a temperature of the algae water solution.

Clause 7. The system of any of the preceding Clauses, wherein the first or second effluent water supply line delivers the stored first or second effluent water from the first or second reservoir to the cultivation vessel to decrease a temperature of the algae water solution below about 35° C.

Clause 8. The system of any of the preceding Clauses, wherein at least one of the first reservoir and the second reservoir is located at an elevation greater than the cultivation vessel.

Clause 9. The system of Clause 8, wherein the stored daytime effluent water is gravity-fed into the cultivation vessel.

Clause 10. The system of Clause 8, wherein the stored nighttime effluent water is gravity-fed into the cultivation vessel.

Clause 11. The system of any of the preceding Clauses, an effluent water conduit that receives effluent water from the cultivation vessel; and a valve positioned in the effluent water conduit to selectively divert the effluent water to the first reservoir or the second reservoir based on a temperature of the effluent water.

Clause 12. The system of any of the preceding Clauses, further comprising a temperature sensor in communication with the effluent water conduit to measure the temperature of the effluent water.

Clause 13. A method comprising: cultivating an algae water solution within a cultivation vessel; extracting a first effluent water from the cultivation vessel and diverting the first effluent water to a first reservoir; optionally extracting a second effluent water and diverting the second effluent water to a second reservoir; and controlling a temperature of the algae water solution by at least one of: delivering the first effluent water to the cultivation vessel; and optionally delivering the second effluent water to the cultivation vessel.

The first effluent water may be a daytime or a nighttime effluent water, and the second effluent water may also be a daytime or nighttime effluent water.

Clause 14. The method of Clause 13, further comprising cultivating the algae water solution within the cultivation vessel within a predetermined period of time.

Clause 15. The method of Clause 14, wherein the predetermined period of time is less than or equal to two days.

Clause 16. The method of any of Clauses 13 to 15, wherein controlling the temperature of the algae water solution further comprises regulating the temperature of the algae water solution to within a range of less than about 30° C. and greater than about 10° C.

Clause 17. The method of any of Clauses 13 to 16, wherein at least one of the first and second reservoirs is located at an elevation greater than the cultivation vessel, the method further comprising gravity-feeding at least one of the first effluent water and the second effluent water into the cultivation vessel.

Clause 18. The method of any of Clauses 13 to 17, further comprising: receiving the effluent water from the cultivation vessel in an effluent water conduit; and selectively diverting the effluent water to the first reservoir or the second reservoir based on the temperature of the effluent water with a valve positioned in the effluent water conduit.

Clause 19. The method of Clause 18, further comprising measuring the temperature of the effluent water with a temperature sensor in communication with the effluent water conduit.

Therefore, the disclosed systems and methods are well adapted to attain the ends and advantages mentioned as well as those that are inherent therein. The particular examples disclosed above are illustrative only, as the teachings of the present disclosure may be modified and practiced in different but equivalent manners apparent to those skilled in the art, having the benefit of the teachings herein. Furthermore, no limitations are intended to the details of construction or design herein shown, other than as described in the claims below. It is, therefore, evident that the particular illustrative examples disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

What is claimed is:

1. A system comprising:
a cultivation vessel containing an algae water solution for cultivation;
a first reservoir in fluid communication with the cultivation vessel to receive and store a first effluent water derived from the algae water solution;
optionally a second reservoir in fluid communication with the cultivation vessel to receive and store a second effluent water derived from the algae water solution;
a first effluent water supply line fluidly coupling the first reservoir to the cultivation vessel to deliver the stored first effluent water to the cultivation vessel; and
optionally a second effluent water supply line fluidly coupling the second reservoir to the cultivation vessel to deliver the stored second effluent water to the cultivation vessel.

2. The system of claim 1, wherein the first effluent water is a daytime effluent water and the second effluent water is a nighttime effluent water.

3. The system of claim 1, wherein the first effluent water is a nighttime effluent water and the second effluent water is a daytime effluent water.

4. The system of claim 1, wherein the cultivation vessel is a closed or open cultivation vessel.

5. The system of claim 1, wherein the cultivation vessel is a photobioreactor.

6. The system of claim 2, wherein the first effluent water supply line delivers the stored daytime effluent water from the first reservoir to the cultivation vessel to increase a temperature of the algae water solution.

7. The system of claim 2, wherein the second effluent water supply line delivers the stored nighttime effluent water from the second reservoir to the cultivation vessel to decrease a temperature of the algae water solution.

8. The system of claim 3, wherein the first effluent water supply line delivers the stored nighttime effluent water from the first reservoir to the cultivation vessel to decrease a temperature of the algae water solution.

9. The system of claim 3, wherein the second effluent water supply line delivers the stored daytime effluent water from the second reservoir to the cultivation vessel to increase a temperature of the algae water solution.

10. The system of claim 2 or 3, wherein the stored daytime effluent water is delivered to the cultivation vessel to increase the temperature of the algae water solution above about 10° C., and the stored nighttime effluent water is delivered to the cultivation vessel to decrease a temperature of the algae water solution to below about 35° C.

11. The system of claim 1, wherein at least one of the first reservoir and the second reservoir is located at an elevation greater than the cultivation vessel.

12. The system of claim 1, wherein the stored effluent water from the first reservoir is gravity-fed into the cultivation vessel.

13. The system of claim 1, wherein the stored effluent water from the second reservoir is gravity-fed into the cultivation vessel.

14. The system of claim 1, further comprising:
an effluent water conduit that receives effluent water from the cultivation vessel; and
a valve positioned in the effluent water conduit to selectively divert the effluent water to the first reservoir or the second reservoir based on a temperature of the effluent water.

15. The system of claim 14, further comprising a temperature sensor in communication with the effluent water conduit to measure the temperature of the effluent water.

16. A method comprising:
cultivating an algae water solution within a cultivation vessel;
extracting a first effluent water from the cultivation vessel and diverting the first effluent water to a first reservoir;
optionally extracting a second effluent water from the cultivation vessel and diverting the second effluent water to a second reservoir; and
controlling a temperature of the algae water solution by:
delivering the first effluent water to the cultivation vessel; and
optionally delivering the second effluent water to the cultivation vessel.

17. The method of claim 16, further comprising cultivating the algae water solution within the cultivation vessel within a predetermined period of time.

18. The method of claim 17, wherein the predetermined period of time is less than or equal to two days.

19. The method of claim 16, wherein the first effluent water is a daytime effluent water and the second effluent water is a nighttime effluent water.

20. The method of claim 16, wherein the first effluent water is a nighttime effluent water and the second effluent water is a daytime effluent water.

21. The method of claim 16, wherein controlling the temperature of the algae water solution further comprises regulating the temperature of the algae water solution to within a range of less than about 30° C. and greater than about 10° C.

22. The method of claim 16, wherein at least one of the first and second reservoirs is located at an elevation greater than the cultivation vessel, the method further comprising gravity-feeding at least one of the first effluent water and the second effluent water into the cultivation vessel.

23. The method of claim 16, further comprising:
receiving the effluent water from the cultivation vessel in an effluent water conduit; and
selectively diverting the effluent water to the first reservoir or the second reservoir based on the temperature of the effluent water with a valve positioned in the effluent water conduit.

24. The method of claim 23, further comprising measuring the temperature of the effluent water with a temperature sensor in communication with the effluent water conduit.
